(19) United States
(12) Patent Application Publication
(10) Pub. No.: US 2005/0107958 A1
(43) Pub. Date: May 19, 2005

(54) Apparatus and method for protein structure comparison and search using 3-dimensional edge histogram

(76) Inventors: Sung Hee Park, Taejon (KR); Soo Jun Park, Seoul (KR); Sung Hun Lee, Seoul (KR); Seon Hee Park, Taejon (KR)

Correspondence Address: JACOBSON, PRICE, HOLMAN & STERN;PROFESSIONAL LIMITED LIABILITY COMPANY, 400 Seventh Street, N.W., Washington, DC, 20004, US

(21) Appl. No.: 10/847,332

(22) Filed: May 18, 2004

(30) Foreign Application Priority Data
Nov. 15, 2003 (KR) 2003-80817

(51) Int. Cl. G06F 7/00; G06F 17/30; G06F 19/00; G01N 33/48; G01N 33/50

(52) U.S. Cl. 702/19; 707/1

(57) ABSTRACT

A protein structure comparison and search apparatus using a 3D edge histogram, the apparatus including: a research client for receiving a query protein from a user to request a search for similar proteins to a protein structure searching server, and outputting the searched result from the searching server; a 3D edge histogram-extracting/storing unit for creating and databasing 3D edge histograms of various proteins; and a protein structure searching server for creating a 3D edge histogram for the query protein, mutually comparing the 3D edge histogram for the query protein with the databased 3D edge histograms of the various proteins to calculate a similarity, and then searching and providing proteins having more than a predetermined similarity.

### BACKGROUND OF THE INVENTION

1. Field of the Invention

The present invention relates to an apparatus and method for searching a similar protein, and more particularly, to an apparatus and method for protein structure comparison and search in which a 3-dimensional (3D) edge histogram, a distribution of each edge pattern is made by atomic or peptide bonding relation in a 3-D structure space of a protein, and proteins having structural similarity with a user's querying protein are detected and provided.

2. Description of the Related Art

A biochemical action within a living body is mostly performed by actions of biomoleculars (i.e., proteins) created by gene revelation. The proteins have respectively proper functions depending on their 3-D structures, that is, their shapes. Accordingly, proteins having a structural similarity perform similar functions, and a search for the proteins having the structural similarity is an important field for an examination for a life phenomenon, curing of a disease, a development for a new medicine, and the like.

In order to perform the search for the similar proteins, many protein representations or descriptors and similarity measures have been proposed for comparison of protein structures.

At an initial time, the similarity measure has been performed depending on comparison of distances between atoms and positions of protein atoms. However, this has a disadvantage in that a calculated amount is generated too much and sensitivity is generated to an error. Accordingly, a method has been proposed in which the similarity is measured using only a position of an alpha carbon of the protein.

Further, a recent study has been made in which the protein is cut as many as the certain number of amino acid and the similarity is measured with an average value on a position of the alpha carbon of the cut amino acid such that its calculation speed is more fast while the disadvantage of the sensitivity to the error is solved.

As another approaching method, a method has been studied in which the proteins are expressed in a format of a vector of a secondary structure included in the protein, and the similarity is measured by using the vector.

### SUMMARY OF THE INVENTION

Accordingly, the present invention is directed to an apparatus and method for protein structure comparison and search using a 3-dimensional edge histogram, which substantially obviates one or more problems due to limitations and disadvantages of the related art.

It is an object of the present invention to provide an apparatus and method for protein structure comparison and search using a 3-dimensional edge histogram for which a new technique is provided where edge patterns in a 3-dimensional structure space can be extracted from a bond distribution or peptide bonding relation of protein atoms to make histograms for the extracted edge patterns and a similarity between the histograms is evaluated to effectively search proteins having structures similar with a query protein, and in which a more fast search is allowed to be performed by incorporating a search considering a whole structure of the protein with a more detailed search.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, there is provided a protein structure comparison and search apparatus using a 3D edge histogram, the apparatus including: a research client for receiving a query protein from a user to request a search for similar proteins to a protein structure searching server, and outputting the searched result from the searching server; a 3D edge histogram-extracting/storing unit for creating and databasing 3D edge histograms of various proteins; and a protein structure searching server for creating a 3D edge histogram for the query protein, mutually comparing the 3D edge histogram for the query protein with the databased 3D edge histograms of the various proteins to calculate a similarity, and then searching and providing proteins having more than a predetermined similarity.

In another aspect of the present invention, there is provided a protein structure comparison and search method using a 3D edge histogram, the method including the steps of: creating and databasing 3D edge histograms for various proteins; creating a 3D edge histogram for a user's querying protein; mutually comparing the histogram of the query protein with the databased histograms of the various proteins to calculate a similarity therebetween; and searching and sequentially providing proteins having more than a predetermined similarity, from a PDB (Protein Data Bank) database.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention, are incorporated in and constitute a part of this application, illustrate embodiments of the invention and together with the description serve to explain the principle of the invention. In the drawings:

FIG. 1 is a block diagram illustrating a protein structure searching system according to the present invention;

FIG. 2 is a flow chart illustrating a procedure in a protein structure searching system according to the present invention;

FIG. 3 is an exemplary view illustrating a Principle Components Analysis (PCA) for a geometric alignment of a protein according to the present invention;

FIG. 4 is an exemplary view illustrating a process of Quantize 3D Volume (QV) of a protein structure according to the present invention;

FIG. 5 is an exemplary view illustrating a 3-dimensional edge pattern of a protein structure according to the present invention; and

FIG. 6 is an exemplary view illustrating a 3-dimensional volume subblock of a protein structure according to the present invention.

### DETAILED DESCRIPTION OF THE INVENTION

Reference will now be made in detail to the preferred embodiments of the present invention, examples of which are illustrated in the accompanying drawings.

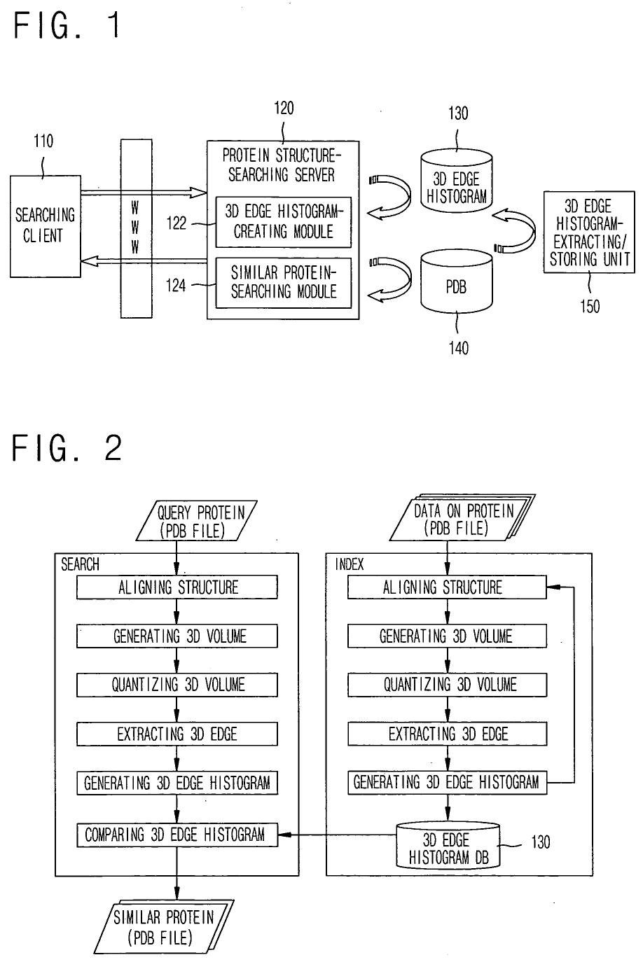

FIG. 1 is a block diagram illustrating a protein structure searching system according to the present invention.

As shown in FIG. 1, the protein structure searching system according to the present invention includes a search client 110 for receiving a query protein from a user, and outputting and displaying the searched result from a protein structure searching server 120; the protein structure searching server 120 for searching and providing proteins having structures similar with the user's querying protein with reference to a large capacity 3D (3-Dimensional) edge histogram 130 and PDB (Protein Data Bank) database 140; a 3D edge histogram-extracting/storing unit 150 for extracting and databasing a 3D edge histogram of each of the proteins from a collection of a large capacity protein data of a PDB; and the like.

Herein, the search client 110 receives a name, etc. of the query protein from the user and connects to the protein structure searching server 120 through a network of WWW internet and the like to request a search for its similar protein, and then sequentially displays the searched proteins transmitted from the searching server 120 depending on its similarity for the user.

Further, the protein structure-searching server 120 is a server for creating the 3D edge histogram for the user's querying protein and then calculating the similarity of the histogram to search and provide the proteins similar with the query protein. The protein structure-searching server 120 includes a 3D edge histogram-creating module 122 and a similar protein-searching module 124.

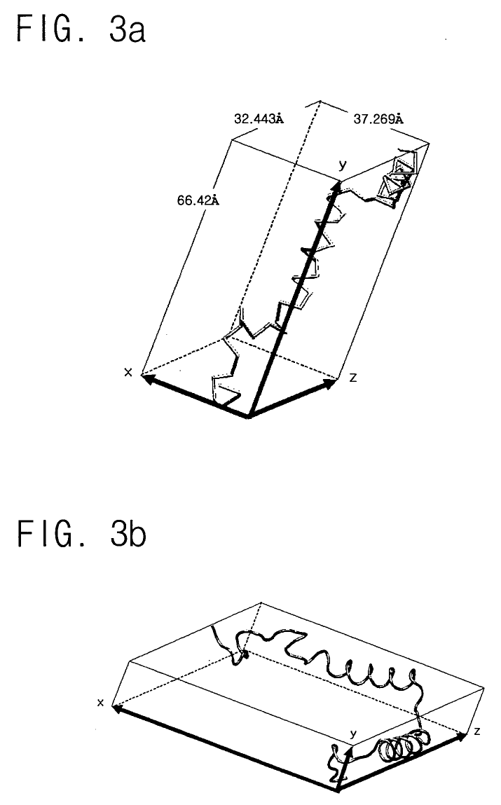

First of all, a procedure of extracting the 3D edge histogram for the user's querying protein from the 3D edge histogram creating module 122 is described with reference to FIGS. 2 to 6.

FIG. 2 is a flow chart illustrating a procedure of creating the 3D edge histogram and databasing the histogram in a protein structure searching system according to the present invention. At a left side of FIG. 2, a procedure of creating the 3D edge histogram for the user's querying protein is schematically illustrated.

As shown in FIG. 2, the 3D edge histogram-creating module 122 performs a 3-dimensional Align Structure (AS) for the query protein.

The 3-D structure alignment is one of very difficult issues. The present invention uses a Principal Components Analysis (PCA) to align an orientation of a 3-dimensional whole protein structure. Herein, the principal components analysis has a geometric meaning in that alignment can be performed with respect to a most long extended axis taken as a main axis.

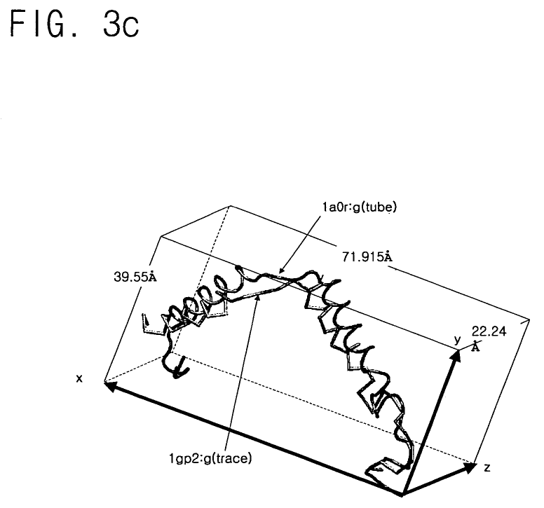

FIG. 3 shows the trace and tube representation of g chains of protein 1gp2 and 1a0r. FIG. 3(a) and (b) presents each conformation of 1gp2 and 1a0r that originally comes from PDB (Protein Data Bank) input file before PCA. As shown in FIG. 3(a), protein 1gp2 lies in order of y>>x>>z(x:37.269 Å, y: 66.42 Å, z:32.443 Å) while protein 1a0r lies in order of x>z>y (x: 64.865 Å, y: 26.187 Å, z:47.82 Å) in FIG. 3(b). FIG. 3(c) displays that after applying PCA. We can find that their conformations have been oriented in order of their principal component after applying PCA. Actually, after applying PCA, 1gp2 is transformed in order of x>y>z(x:71.915 Å, y: 33.785 Å, z:14.029 Å) and also 1a0r is transformed in order of x>y>z x:69.492 Å, y: 39.55 Å, z:22.24 Å).

If the 3-D structure alignment performed as described above, the 3D edge histogram-creating module 122 performs a process of a Generating 3D Volume (GV). In order to obtain an atomic bond distribution, a 3-D space is digitalized at a certain size and sampled at a certain distance.

For this, information on 3-D positions of atoms is read from the protein structure information changed through the Principal Components Analysis. Additionally, bond information (atomic or peptide bonding relation) is created from the read position information, and the created bond information is used to perform a spatial sampling for generating a 3-D volume. By the spatial sampling, the 3-D structure space of the protein is divided into a plurality of voxels (the voxel is a compound word of a volume and a pixel).

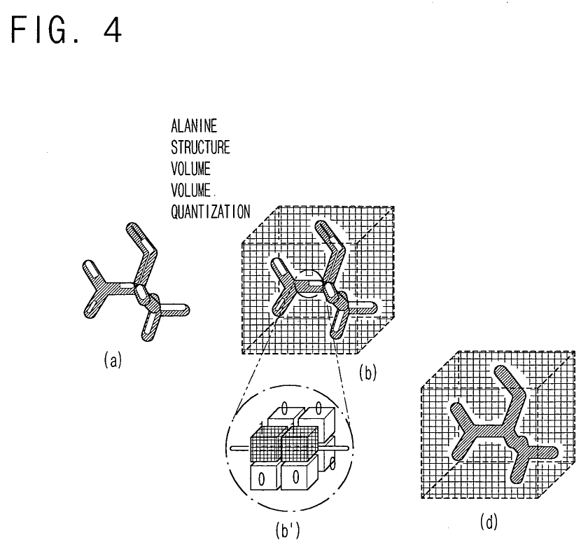

Additionally, the 3D edge histogram-creating module 122 performs a process of a Quantizing 3D Volume (QV). The 3-D structure space of the query protein is digitalized into the voxels. A case that the bond passes through the voxel is represented as “1”, and a case that the bond does not pass through the voxel is represented as “0”. That is, a whole 3-dimensional structure space is binary-quantized. FIG. 4 is a view illustrating the process of the Quantizing 3D Volume (QV) of the protein structure according to the present invention. In this FIG. 4(b′), the voxel through which the bond passes is expressed with a checked-pattern surface, and an otherwise voxel is expressed with a white-colored surface.

Through the above quantization process, an edge is created between a bond passing part and a non-passing part as shown in FIG. 4. The 3D edge histogram-creating module 122 performs a process of Extracting 3D Edge (EE) depending on a pattern of the edge therebetween.

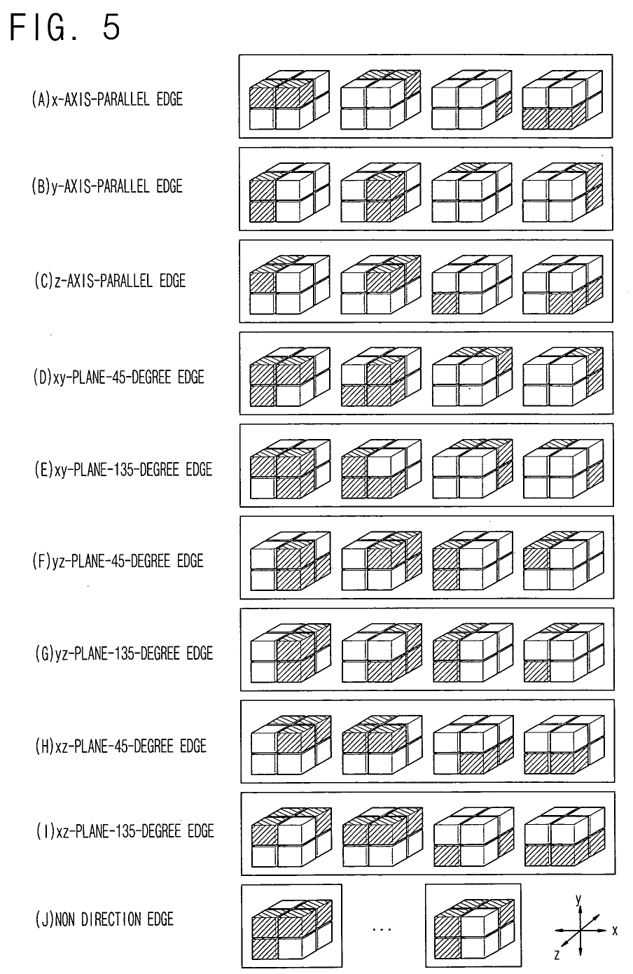

In this procedure of an embodiment of the present invention, 10 kinds of 3-D edge patterns are defined to extract the edge pattern in a unit of eight voxels as shown in FIG. 5.

Each of the edge patterns is described with reference to FIG. 5. The most upper edge pattern has four cases having edges parallel to x-axis, and is defined as “x-axis-parallel-edge pattern.” Further, edge patterns parallel to y-axis and z-axis can also respectively have four cases such as the x-axis-parallel-edge pattern, and can be respectively defined as “y-axis-parallel-edge pattern” and “z-axis-parallel-edge pattern.”

Further, a “45-degree edge pattern” and a “135-degree edge pattern” can be obtained with respect to xy-plane, xz-plane and yz-plane. Lastly, a “non direction edge pattern” not having a direction can be defined. Accordingly, 10 kinds of edge patterns can be defined in total.

On the other hand, the 3D edge histogram creating module 122 performs a distribution of 3-D edges, that is, a process of Making 3D edge Histogram (MH) on basis of a result extracted from the process of Extracting 3D Edge (EE).

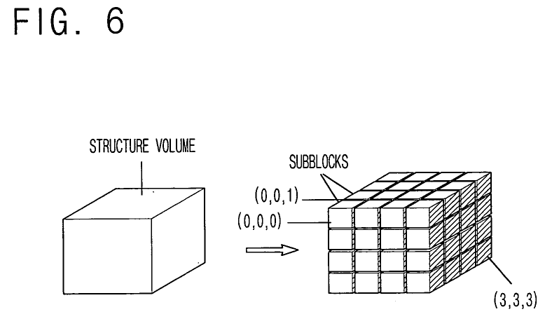

For this, as shown in FIG. 6, a structure volume is needed to be largely sub-divided into 2×2×2 or 4×4×4 for each axis so that each subdivided sub-volume may able to contain 3D edges according to their patterns which have extracted during the 3D edge extraction process. The 3D volume is divided into 2×2×2 volumes for a search considering a whole protein shape, and the 3D volume is divided into 4×4×4 volumes for a more detailed search such that they are compared with one another.

The above divided sub-structure volume is called subblock. The above-defined 10 kinds of edge patterns are extracted from respective subblocks. That is, the 3D edge histogram is made through confirmation of the number of the edge patterns included in respective subblocks. Since each of the subblocks is comprised of a plurality of voxels, a plurality of the edge patterns extracted from all 2×2×2 voxel volumes' (Referring to FIG. 4(b′)), exists within one subblock. As a distribution of each of the edge patterns (the number) included within each of the subblocks, the 3D edge histogram is made.

In case that the structure volume is divided into 2×2×2 subblocks, a total number of histogram bins is 80 obtained by multiplying the number of the subblock (8) by the number of the edge pattern (10). In case that the structure volume is divided into 4×4×4 subblocks, 640 histogram bins are obtained.

Table 1 below illustrates Semantics of 3D edge histogram bins in case of the 4×4×4 subblocks.

TABLE 1

| Bins | Semantics |
|---|---|
| 3D_Edge[0] | X-axis parallel edge of subblock(0, 0, 0) |
| 3D_Edge[1] | Y-axis parallel edge of subblock(0, 0, 0) |
| 3D_Edge[2] | Z-axis parallel edge of subblock(0, 0, 0) |
| 3D_Edge[3] | Xy-plane 45 degree edge of subblock(0, 0, 0) |
| 3D_Edge[4] | Xy-plane 135 degree edge of subblock(0, 0, 0) |
| 3D_Edge[5] | Xz-plane 45 degree edge of subblock(0, 0, 0) |
| 3D_Edge[6] | Xz-plane 45 degree edge of subblock(0, 0, 0) |
| 3D_Edge[7] | Yz-plane 45 degree edge of subblock(0, 0, 0) |
| 3D_Edge[8] | Yz-plane 45 degree edge of subblock(0, 0, 0) |
| 3D_Edge[9] | Non-directional edge of subblock(0, 0, 0) |
| 3D_Edge[10] | X-axis parallel edge of subblock(0, 0, 1) |
| . | . |
| . | . |
| . | . |
| 3D_Edge[638] | Yz-plane 45 degree edge of subblock(3, 3, 3) |
| 3D_Edge[639] | Non-directional edge of subblock(3, 3, 3) |

Further, each value of the histogram bins represents the number of the edge patterns included within the corresponding subblock.

On the other hand, the similar protein searching module 124 calculates the similarity between the made histogram of the query protein and the protein histograms stored in the 3D edge histogram DB to confirm the similar proteins, and extracts information on the corresponding proteins from the PDB 140 to provide the extracted information for the client 110.

Herein, on basis of Euclidean distance concept, the larger the similarity of the 3D edge histogram is, the smaller a distance value of the 3D edge histogram is. That is, the distance value between the histogram of the 3D edge histogram DB 130 and the histogram of the query protein in the dimensional space having each of the histogram bins is used.

The above similarity calculation can be executed using various methods depending on those skilled in the art. In calculating the similarity, a weighted value can be applied. There are a method of calculating all of the histogram bins by using the same weighted value, and a method of calculating using different weighted values provided depending on importance of each subblock or each bin.

Further, even in case that the similarity is determined, the similarity for an entire 3-dimensional structure is calculated, or the similarity is calculated every subblock and then the calculated similarities are added to one another to calculate a total similarity. Further, the similarity is compared every subblock, or the subblocks having a maximum distance value or a minimum distance value are mutually compared with one another such that the similar proteins can be searched.

In the meantime, in order to perform the more fast search from the large protein structure database, the similar protein searching module 124 performs filtering for the proteins having an entire shape similar with the user's querying protein through the similarity evaluation between histogram data according to division for the 2×2×2 subblocks, and then performs the more detailed search for the filtered proteins through the similarity evaluation of the 3D edge histogram according to division for the 2×2×2 subblocks.

Meanwhile, the 3D edge histogram-extracting/storing unit 150 is a device for confirming structural information on various proteins from the PDB database 140, and creating and databasing their 3D edge histograms. The 3D edge histogram-extracting/storing unit 150 performs the same process as the 3D edge histogram creating module 122 of the protein structure searching server 120 to make the 3D edge histogram of each protein. Additionally, it stores the extracted 3D edge histogram in a file every protein to database the stored 3D edge histogram in the 3D edge histogram DB 130.

At this time, it is desirable that the histogram data according to the division for the 2×2×2 subblocks and the histogram data according to the division for the 4×4×4 subblocks are respectively created and databased for each protein so as to perform the more fast search.

The protein structure comparison and search method using the 3D edge histogram can be stored in a recording media that can be read using a computer. The recording media includes various recording medias having program and data stored therein to be able to be read using a computer system. For example, there are ROM (Read Only Memory), RAM (Random Access Memory), CD (Compact Disk)-ROM, DVD (Digital Video Disk)-ROM, a magnetic tape, a floppy disk, an optic data storage device and the like. Further, the recording media is dispersively disposed in the computer system connected over a network to store and execute a code readable by the computer in a dispersion way.

As described above, the protein structure comparison and search method using the 3D edge histogram according to the present invention provides a new technique in which the edge patterns based on the bond distribution of the protein atoms are extracted to make their histograms in the 3-D structure space, and the similarity between the made histograms is evaluated such that the proteins having the structure similar with the query protein can be effectively searched on Web and the like.

Further, the present invention incorporates the search based on the entire structure with the more detailed search and performs the incorporated search such that the fast search can be achieved for the large PDB, and provides a very effective research before a more precise structure comparison in a prescreening process.

It will be apparent to those skilled in the art that various modifications and variations can be made in the present invention. Thus, it is intended that the present invention covers the modifications and variations of this invention provided they come within the scope of the appended claims and their equivalents.

1. A protein structure comparison and search apparatus using a 3D edge histogram, the apparatus comprising:
a research client for receiving a query protein from a user to request a search for similar proteins to a protein structure searching server, and outputting the searched result from the searching server;
a 3D edge histogram-extracting/storing unit for creating and databasing 3D edge histograms of various proteins; and
a protein structure searching server for creating a 3D edge histogram for the query protein, mutually comparing the 3D edge histogram for the query protein with the databased 3D edge histograms of the various proteins to calculate a similarity, and then searching and providing proteins having more than a predetermined similarity.

2. The apparatus of claim 1, wherein the protein structure searching server comprises:
a 3D edge histogram creating module for treating atomic or peptide bonding relation of the query protein as an edge, and creating the 3D edge histogram using a distribution of each edge pattern in a 3-D structure space of the protein; and
a similar protein searching module for mutually comparing the histogram of the query protein with the histograms of the various proteins to calculate the similarity, and searching and providing the similar proteins in order of a larger similarity.

3. The apparatus of claim 1, wherein the 3D edge histogram-extracting/storing unit and the protein structure searching server make the 3D edge histogram for a target protein by performing the steps of:
(a) performing a 3-D structure alignment for the target protein;
(b) performing a spatial sampling for the 3-D structure alignment of the target protein to generate a 3D volume of the target protein comprised of a plurality of voxels;
(c) creating atomic bond information on the target protein, and quantizing the 3D volume of the target protein by “0” or “1” depending on whether or not a bond passes through the voxels;
(d) dividing a 3-D structure volume of the target protein into a plurality of subblocks; and
(e) defining edge patterns depending on a format quantized at certain voxels, and creating the 3D edge histogram of the target protein by using a distribution of the edge pattern included within each of the subblocks.

4. The apparatus of claim 3, wherein the 3D edge histogram-extracting/storing unit and the protein structure searching server perform the step (a) by aligning an orientation of a 3-dimensional structure of the target protein through a Principal Components Analysis (PCA) having a longest axis as a geometric main axis.

5. The apparatus of claim 3, wherein the 3D edge histogram-extracting/storing unit and the protein structure searching server perform the step (d) by dividing the 3-D structure volume into 8 subblocks of 2×2×2 for a search considering an entire shape of the target protein, and dividing the 3-D structure volume into 64 subblocks of 4×4×4 for a more detailed search.

6. The apparatus of claim 3, wherein the 3D edge histogram-extracting/storing unit and the protein structure searching server perform the step (e) by defining 10 kinds of 3-D edge patterns of “x-axis-parallel edge pattern”, “y-axis-parallel edge pattern”, “z-axis-parallel edge pattern”, “45-degree edge pattern” and “135-degree edge pattern” for each of xy plane, xz plane and yz plane, and “non direction edge pattern” depending on a quantization format of a block comprised of 2×2×2 voxel volume.

7. The apparatus of claim 2, wherein the similar protein searching module calculates a distance value of a histogram between a query protein and its comparison protein on basis of Euclidean distance concept to yield a similarity.

8. The apparatus of claim 7, wherein the similar protein searching module provides different weighted values depending on importance of each of the subblocks or each of histogram bins to calculate the similarity.

9. The apparatus of claim 7, wherein the similar protein searching module calculates the similarity between the query protein and its comparison protein through any one method among a method for calculating a similarity using a distance value of both histograms for an entire 3-D structure, a method for calculating a distance value every subblock and then adding the calculated distance value to yield a total similarity, and a method for calculating a distance value every subblock and then yielding a similarity using minimal or maximal value of the calculated distance value.

10. The apparatus of claim 5, wherein the similar protein searching module extracts proteins having entire shapes similar with a user's querying protein through a similarity evaluation of the histogram according to a first subblock division, and then searches similar proteins among the extracted proteins through a similarity evaluation of the histogram according to a more detailed second subblock division.

11. A protein structure comparison and search method using a 3D edge histogram, the method comprising the steps of:
creating and databasing 3D edge histograms for various proteins;
creating a 3D edge histogram for a user's querying protein;
mutually comparing the histogram of the query protein with the databased histograms of the various proteins to calculate a similarity therebetween; and
searching and sequentially providing proteins having more than a predetermined similarity, from a PDB (Protein Data Bank) database.

12. The method of claim 11, wherein the 3D edge histogram for the target protein is made in the steps (a) and (b) by treating atomic or peptide bonding relation of a target protein as an edge, and creating the 3D edge histogram using a distribution of each edge pattern in a 3-D structure volume.

13. The method of claim 11, wherein the 3D edge histogram for the target protein is made in the steps (a) and (b) by performing the steps of:
performing a 3-D structure alignment for the target protein;
performing a spatial sampling for the 3-D structure alignment of the target protein to generate a 3D volume of the target protein comprised of a plurality of voxels;
creating atomic bond information on the target protein, and quantizing the 3D volume of the target protein by “0” or “1” depending on whether or not a bond passes through the voxels;
dividing a 3-D structure volume of the target protein into a plurality of subblocks; and
defining edge patterns depending on a format quantized at certain voxels, and creating the 3D edge histogram of the target protein using a distribution of the edge pattern included within each of the subblocks.

14. The method of claim 13, wherein the structure alignment step is performed by aligning an orientation of a 3-D structure of the target protein through a Principal Components Analysis (PCA) using a longest axis as a geometric main axis.

15. The method of claim 11, wherein the subblock dividing step is performed by performing a first subblock division for a search considering an entire shape of the target protein, and again performing a second subblock division of the first subblocks for a more detailed search, and the 3D edge histogram-creating step is performed by respectively creating the histogram of the target protein according to the first subblock division and the histogram of the target protein according to the second subblock division.

16. The method of claim 15, wherein the (d) step is performed by extracting the proteins having entire shapes similar with the user's querying protein through a similarity evaluation of the histogram according to the first subblock division, and then searching and providing similar proteins among the extracted proteins through a similarity evaluation of the histogram according to the second subblock division.

17. The method of claim 13, wherein the edge pattern is defined in the 3D edge histogram creating step by defining 10 kinds of 3-dimensional edge patterns of “x-axis-parallel edge pattern”, “y-axis-parallel edge pattern”, “z-axis-parallel edge pattern”, “45-degree edge pattern” and “135-degree edge pattern” for each of xy plane, xz plane and yz plane, and “non direction edge pattern” depending on a quantization format of a block comprised of 2×2×2 voxel volume.

18. The method of claim 11, wherein the step (c) is performed by calculating the distance value of the histogram between the query protein and its comparison protein on basis of Euclidean distance concept to yield the similarity, and providing different weighted values depending on importance of each of the subblocks or each of the histogram bins to calculate the similarity.

19. The method of claim 11, wherein the step (c) is performed by calculating the similarity between the query protein and its comparison protein through any one method among a method for calculating a similarity using a distance value of both histograms for an entire 3-D structure, a method for calculating a distance value every subblock and then adding the calculated distance value to yield a total similarity, and a method for calculating a distance value every subblock and then yielding a similarity using minimal or maximal value of the calculated distance value.
